(19) United States
(12) Patent Application Publication
(10) Pub. No.: US 2021/0308046 A1
(43) Pub. Date: Oct. 7, 2021

(54) Method, Apparatus and Composition for Preventing Infection From Coronavirus, Limiting its Spread and Treatment Thereof

(71) Applicants: David MICHAELI, Ashkelon (IL); Menashe Michaeli, Ashkelon (IL)

(72) Inventors: David MICHAELI, Ashkelon (IL); Menashe Michaeli, Ashkelon (IL)

(21) Appl. No.: 16/841,776

(22) Filed: Apr. 7, 2020

Related U.S. Application Data
(60) Provisional application No. 63/003,304, filed on Apr. 1, 2020.

(51) Int. Cl. A61K 9/00; A61M 15/00; A61K 31/4706; A61K 33/20; A61K 31/045

(52) U.S. Cl. CPC A61K 9/0078; A61M 15/0065; A61M 15/009; A61K 45/06; A61K 33/20; A61K 31/045; A61K 31/4706

(57) ABSTRACT

A nebulizer is used to prevent infection by coronavirus by nebulizing a combination of hydrogenated water and ethyl alcohol, one or both of which have a counterclockwise spin polarization, into a vapor-aerosol mixture for inhalation by a patient. A storage container of helium adjacent to or in communication with an inside of the nebulizer increases partial pressure of the vapor-aerosol mixture during usage. The nebulizer may also nebulize into the vapor-aerosol mixture, one of the following third ingredient (a) hydrogen peroxide, (b) a low dosage of hydroxychloroquine or chloroquine (c) a very low dosage of a mixture of sodium chlorite with food-grade citric acid solution, (d) antibiotics. The third ingredient may vary to thwart development of resistance. A nebulized vapor-aerosol mixture produced by nebulizing a combination of ingredients is presented. A method of delivering a combination of ingredients directly to the lungs of a subject, at times bolstered by helium.

### FIELD OF THE INVENTION

The invention relates to a method, composition and/or apparatus for prevention of infection—and treatment of infection—by viruses such as coronavirus, including COVID-19, and for limiting the virus from spreading among the population.

### BACKGROUND

One mode of transmission of the coronavirus COVID-19 is that when people talk to one another, one person breathes in the air that the other person breathes out during their conversation. When that happens, the coronavirus that may exist in the exhaled air of the infected person may enter the lungs of the other person. However, the virus does not penetrate the cells of the lungs yet. After several hours, it penetrates the alveolar epithelial cells of the lungs. This happens when the protein on the outer shell of the coronavirus, called the spike protein, binds to the ACE2 (angiotensin converting enzyme) of the lung cells. ACE2 is a membrane protein with its enzymatically active domain exposed on the surface of cells in lungs and other tissues.

### SUMMARY OF THE EMBODIMENTS

One aspect is a method of preventing infection of a mammalian subject by a coronavirus, comprising administering to the subject, by having the subject inhale, an effective dosage of a vapor-aerosol mixture emitted by a nebulizer derived from the following quantities placed into the nebulizer: hydrogenated water, and ethyl alcohol between 40% and 80%, wherein at least one of the ethyl alcohol and hydrogenated water has a counterclockwise spin polarization.

In some embodiments, the mixture also contains one of the following: (a) hydrogen peroxide, (b) hydroxychloroquine or chloroquine, (c) a yellow derivative of a mixture of sodium chlorite with citric acid solution, (d) antibiotics. In some embodiments, the mixture also contains one of the following: (a) hydrogen peroxide, (b) hydroxychloroquine or chloroquine, (c) a yellow derivative of a mixture of sodium chlorite with citric acid solution, (d) antibiotics, (e) essential oils.

In some embodiments, the hydroxychloroquine or chloroquine is between 50 and 70 mgs for each milliliter of hydrogenated water placed into the nebulizer.

In some embodiments, the mixture of sodium chlorite with citric acid solution is in a quantity of 0.03 to 0.1 milliliters.

In some embodiments, the method further provides a flow of helium into the nebulizer during the inhalation of the VAM emitted by the nebulizer.

In some embodiments, the hydrogenated water and the ethyl alcohol each have a counterclockwise spin polarization.

In some embodiments, for each milliliter of hydrogenated water in the nebulizer, the ethyl alcohol is between 0.05 and 0.3 milliliters.

In some embodiments, the administering of the treatment is performed at least three times a day for at least 10 days.

In some embodiments, the administering is performed such that each day of the administering of the treatment, the mixture also contains one of (a) hydrogen peroxide, (b) hydroxychloroquine or chloroquine, (c) a yellow derivative of a mixture of sodium chlorite with citric acid solution, (d) antibiotics, wherein the one of the (a) hydrogen peroxide, (b) hydroxychloroquine or chloroquine, (c) yellow derivative of the mixture of sodium chlorite with citric acid solution, (d) antibiotics, varies daily or varies with each treatment.

Another aspect is an apparatus configured to prevent infection of a mammalian subject by a coronavirus, the apparatus comprising a nebulizer holding ethyl alcohol between 40% and 80% and hydrogenated water and configured to nebulize into a vapor-aerosol mixture the ethyl alcohol and the hydrogenated water, wherein at least one of the ethyl alcohol and hydrogenated water has a counterclockwise spin polarization.

In some embodiments, the nebulizer is configured to also hold, and nebulize into the vapor-aerosol mixture, one of the following (a) hydrogen peroxide, (b) hydroxychloroquine or chloroquine (c) a yellow derivative of a mixture of sodium chlorite with citric acid solution, (d) antibiotics.

In some embodiments, the nebulizer further comprises a storage container of helium such that the helium is configured to be emitted from the storage container in a direction so as to mix with the vapor-aerosol mixture emitted from the nebulizer and jointly enter a lungs of a user.

In some embodiments, the apparatus further comprises a storage container of helium such that the helium is in communication with the nebulizer through an inlet of the nebulizer. In some embodiments, the storage container is a balloon adjacent the nebulizer.

In some embodiments, the nebulizer also holds, and is configured to nebulize into the vapor-aerosol mixture, one of the following (a) hydrogen peroxide, (b) hydroxychloroquine or chloroquine (c) a yellow derivative of a mixture of sodium chlorite with citric acid solution, (d) antibiotics. In some embodiments, the hydroxychloroquine or chloroquine is between 50 and 70 mgs for each milliliter of hydrogenated water in the nebulizer. In some embodiments, a quantity of the yellow derivative of the mixture of sodium chlorite with citric acid solution is between 0.03 and 0.1 milliliters.

In some embodiments, each of the ethyl alcohol and the hydrogenated water has a counterclockwise spin polarization.

In some embodiments, the nebulizer holds between 0.05 and 0.3 milliliters of the ethyl alcohol for each milliliter of the hydrogenated water in the nebulizer. In some embodiments, the nebulizer holds 50-70 mgs of hydroxychloroquine or chloroquine. In some other embodiments, the nebulizer holds between 0.03 and 0.1 milliliters of a yellow derivative of a mixture of sodium chlorite with citric acid solution in the nebulizer.

Yet another aspect is a nebulized vapor-aerosol mixture produced by nebulizing a combination of ingredients, the mixture under pressure to move through the air, the combination comprising hydrogenated water and ethyl alcohol and having a proportion such that for each milliliter of hydrogenated water in the combination, there is between 0.05 and 0.3 milliliters of ethyl alcohol of a solution between 40% and 80% ethyl alcohol, wherein at least one of the hydrogenated water and the ethyl alcohol has a counterclockwise spin polarization.

In some embodiments, the hydrogenated water has a counterclockwise spin polarization.

In some embodiments, the combination further includes for each milliliter of hydrogenated water, one of the following: (i) between 0.03 and 0.1 milliliters of a derivative of a mixture of sodium chlorite with citric acid solution; (ii) 50-70 mgs of hydroxychloroquine or chloroquine; (iii) 0.025 to 0.1 milliliters of hydrogen peroxide that is between 20% and 35% hydrogen peroxide; (iv) between 30 mg and 150 mg of an antibiotic.

In some embodiments, the mixture is also under pressure to move from helium under pressure adjacent the mixture.

Yet still another aspect is a method of delivering a combination of ingredients directly to the lungs of a live mammalian subject, comprising nebulizing the combination of ingredients in a nebulizer to produce a nebulized vapor-aerosol mixture, the combination having a proportion such that for each milliliter of hydrogenated water in the combination, there is between 0.05 and 0.3 milliliters of ethyl alcohol of a solution between 40% and 80% ethyl alcohol, wherein at least one of the hydrogenated water and the ethyl alcohol has a counterclockwise spin polarization; and emitting the nebulized vapor-aerosol mixture in a direction of a face of the subject so as to enter the lungs of the subject.

The method, in some embodiments, further comprises providing a thrust of helium, the helium under pressure in a storage container, in a particular direction so as to mix with the vapor-aerosol mixture and drive the vapor-aerosol mixture in the direction of the face of the user. In some versions, the storage container is in communication with an inside of the nebulizer.

In some embodiments of the method, the combination further includes for each milliliter of hydrogenated water in the nebulizer, one of the following: (i) between 0.025 and 0.15 milliliters of a derivative of a mixture of sodium chlorite with citric acid solution; (ii) 40-80 mgs of hydroxychloroquine or chloroquine; (iii) 0.025 to 0.1 milliliters of hydrogen peroxide that is between 20% and 35% hydrogen peroxide; (iv) between 30 mg and 150 mg of an antibiotic

These and other features, aspects and advantages of the invention will become better understood with reference to the following drawings, descriptions and claims.

### BRIEF DESCRIPTION OF THE DRAWINGS

Various embodiments are herein described, by way of example only, with reference to the accompanying drawings, wherein:

FIG. 1 is a schematic view of a nebulizer apparatus, used in accordance with one embodiment of the invention;

FIG. 2 is a photo of a coronavirus showing spike protein;

FIG. 3 is a schematic illustration of preparation of hydrogen enriched water in accordance with use of one particular embodiment of the invention;

FIG. 4 shows a method of making right-side or counterclockwise ethyl alcohol (or hydrogenated water);

FIG. 5 is a flow chart showing one particular embodiment of a method; and

FIG. 6 is a flow chart showing another embodiment of a method.

### DETAILED DESCRIPTION OF THE EMBODIMENTS

The following detailed description is of the best currently contemplated modes of carrying out the invention. The description is not to be taken in a limiting sense, but is made merely for the purpose of illustrating the general principles of the invention, since the scope of the invention is best defined by the appended claims.

There are currently a variety of approaches for effective coronavirus therapy. One approach is to use antivirals, small molecules that interfere with viral RNA polymerases. Another approach is immune-modulating therapies designed to modify and dampen overactive and adverse immune response. A still further approach is antibody therapies, namely using blocking antibodies against COVID-19 engineered or collected from recovered individuals. The antibodies are designed to target the spike protein and block it, inhibiting cellular adhesion and infection, as well as inducing immune response. A yet still further approach is vaccines—inducing adaptive immune response against viral determinants. The vaccine induces the expression of selected viral proteins to induce adaptive immunity. These vaccines have not yet been shown to work and are not available yet and the US government suggests it would take a year or so for any of them to be available. A further approach is viral entry inhibitors like chloroquine (CQ) or Hydroxychloroquine. Originally designed to treat malaria, these drugs were repurposed to treat SARS-CoV-2. There are several mechanisms of action such as by preventing the viral entry step, due to endosomal pH modulation (increase). Additionally, the drug supposedly modifies ACE-2 glycosylation, reducing viral spike protein binding. One company, Sorrento, has developed a fusion protein in which the cellular receptor ACE2 is fused to the Fc domain of immunoglobulin. This fusion protein is designed to bind the SARS-CoV-2 spike protein and inhibit the binding of virus to epithelial cells in the respiratory system.

These therapies are new and experimental and may be very expensive. These known methods of influencing the spike protein are immunological rather than chemical or physical.

Currently, there is no vaccine available. It is estimated that this will take at least a year. A medicine that can deal with the coronavirus would change things dramatically because it would make the heath care crisis (and the resulting economic crisis) much more manageable until there is a vaccine.

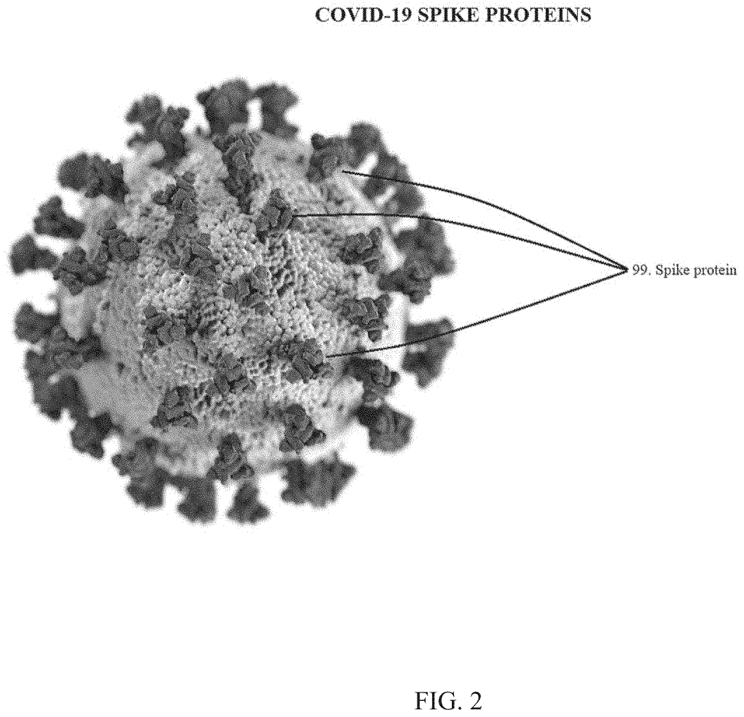

Certain embodiments of the invention influence spike proteins 99 (FIG. 2) of the coronavirus in a chemical manner in order to neutralize the spike protein of the virus. Certain embodiments of the invention neutralize the spike protein 99 and as a result the coronavirus loses the ability to bind with ACE2 of the lung cells. The chemical approach has an advantage over the immunological approach in certain embodiments since unlike the immunological approach which make take up to a week for the body to produce the desired immunological (antibody) response, the chemical approach operates immediately—provides immediate relief—in some embodiments. In some embodiments the chemical approach is also cost effective, widely available and non-invasive.

Certain embodiments influence both the spike protein and the ACE2 enzyme, thereby doubly inhibiting the binding of the spike protein to the ACE2 membrane protein, which is the mechanism of penetration by the virus into the cell to replicate itself and cause damage.

Certain embodiments of the invention provide a method, apparatus and/or composition with a unique delivery system for administering a treatment for prevention of damaging (infecting) exposure to coronavirus, for example COVID-19. In some embodiments, the delivery system involves nebulizing a combination of ingredients and delivering the combination in a form of a nebulized vapor-aerosol mixture directly to the lungs. Since it is delivered directly to the lungs, the combination utilizes low dosages of the ingredients in the combination. In certain implementations, the nebulized vapor-aerosol mixture is delivered under pressure, for example using helium under pressure (for example helium stored in a small balloon).

The principles and operation of a Method, Apparatus and Composition for Preventing Infection From Coronavirus, Limiting its Spread and Treatment Thereof may be better understood with reference to the drawings and the accompanying description.

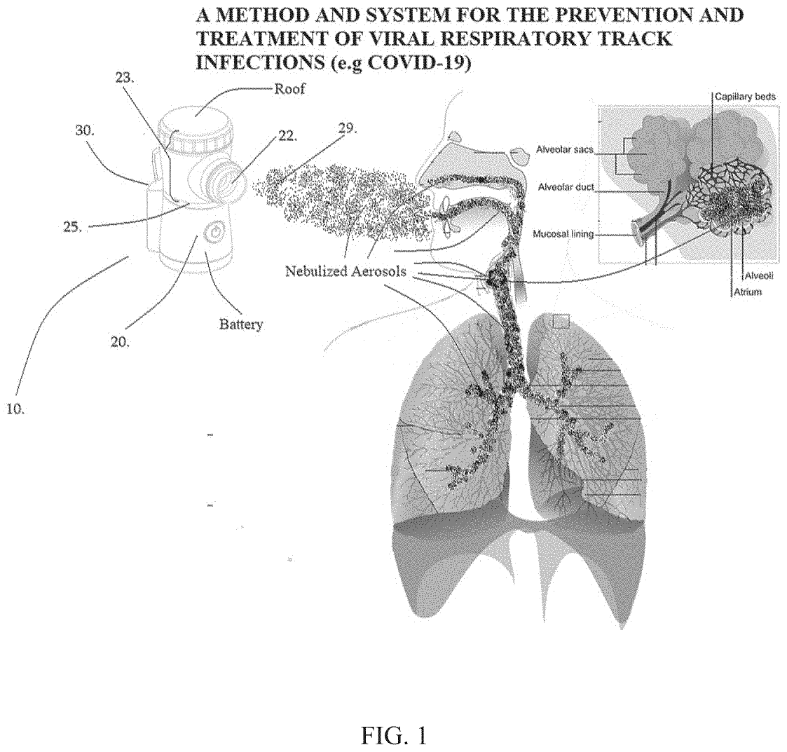

In one particular embodiment, as shown in FIG. 1, a nebulizer apparatus 10 includes a nebulizer 20 that is configured to hold, and in accordance with certain embodiments is filled with, certain ingredients and then turned on (to nebulize the ingredients) so that a person can inhale the ingredients emitted by the nebulizer 20 into their lungs. In some cases, this inhalation occurs three times a day. In some embodiments, treatment regimen continues for about 10 days, or as many days as the subject considers themselves to be vulnerable to exposure to the virus, for example by meeting others.

Below is a group of ingredients to be mixed together in the nebulizer 20, for example to generate a vapor-aerosol mixture (“VAM”). Although the list of ingredients is presented sequentially, in other embodiments, the sequence presented below is not followed or is not followed completely or not in all respects.

In general, when the term “drops” or “drop” is used herein, the reference is to small drops. In certain embodiments, for reference purposes this refers to drops of about one-twentieth of one cc (one milliliter). However, it is not the intention to place any limitation on the actual size of the drops used in any of the embodiments; rather this definition of the “drops” is merely a reference point for calculating the quantity of the ingredient.

In one non-limiting embodiment, the nebulizer 20 has a capacity of between 3-10 milliliters, for example five milliliters. Nebulizer 20 may be battery operated although this is not a limitation. The proportions of the ingredients presented below are geared for a nebulizer 20 having a capacity of 5 milliliters but larger capacity nebulizers (or smaller) may be used. Moreover, if one uses a larger capacity nebulizer, one need not fill up the nebulizer 20 to its capacity. Accordingly, the magnitude of the ingredients may remain the same even with a larger capacity nebulizer 20 or they may be increased proportionately (with the exception of the sodium chlorite, if present, which should not be increased proportionately in certain embodiments).

In certain embodiments, the proportion between the different ingredients is maintained. In some embodiments, the one exception is the yellow derivative of the acidified sodium chlorite mixture (in versions where it is present). The maximum dosage of the sodium chlorite mixture should be maintained at no greater than about 0.1 milliliters (about two small drops) even if the other ingredients are increased (for example even if the hydrogenated water exceeds one milliliter or even if the ethyl alcohol exceeds 0.2 milliliters. In the event that the magnitude of the quantities of the ingredients are reduced, though, the magnitude of the quantity of the sodium chlorite mixture (in embodiments where it is present) should also be reduced.

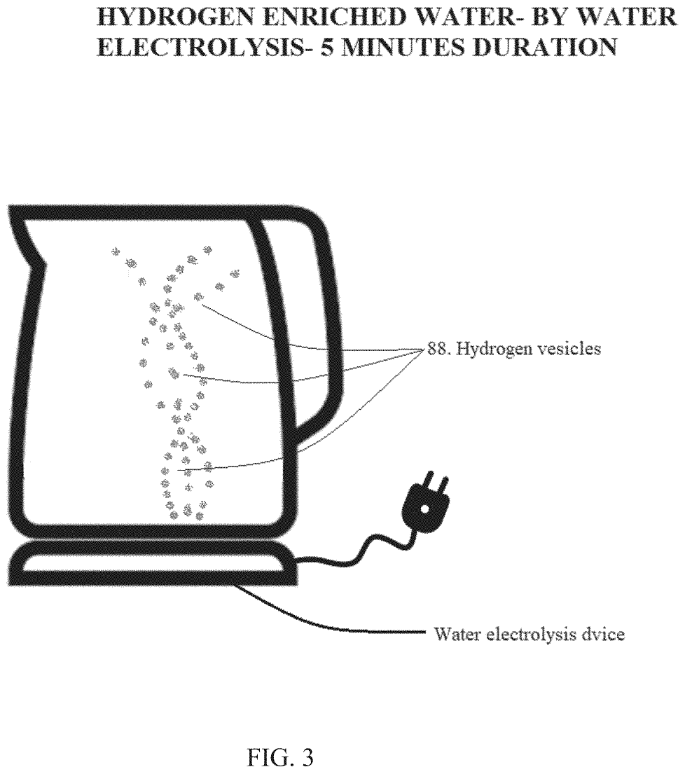

One typically adds water and ethyl alcohol into the nebulizer 20 first before adding other ingredients, although this is not an absolute limitation. One would typically begin by adding hydrogenated water into the nebulizer 20 since that is the largest quantity. In some embodiments, one adds about one milliliter of hydrogenated water, which is water enriched with hydrogen, where “about” means plus or minus 20%. In other embodiments, “about” means plus or minus 15%, or plus or minus 10% or plus or minus 5% or plus or minus 3%. As shown in FIG. 3, applying electrolysis for about 5 minutes is one way of making hydrogenated water or hydrogen enriched water. In certain embodiments, the minimum amount of hydrogenated water used to nebulize the mixture is half a milliliter. In certain embodiments, the minimum amount of hydrogenated water used to nebulize the mixture is three-quarters of a milliliter or is one milliliter (or it may be more).

The presence of hydrogenated water in the combination of ingredients to be nebulized into the vapor-aerosol mixture (VAM) enhances the penetration of the vapor-aerosol mixture 29 (FIG. 1) into the lungs of the user/subject/patient because of the small particles 88 (FIG. 3) of hydrogen in the hydrogenated water. Hydrogenated water is considered GRAS (generally accepted as safe) by the U.S. FDA.

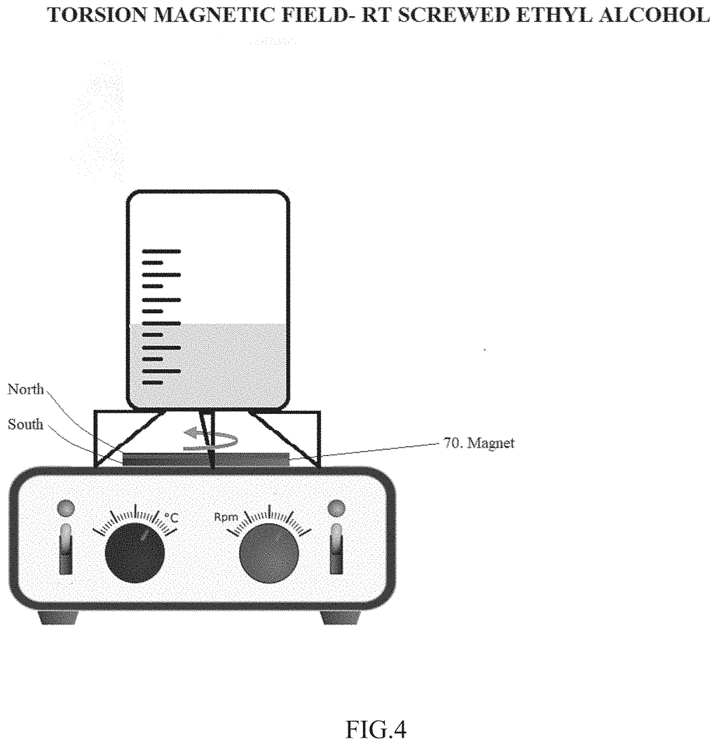

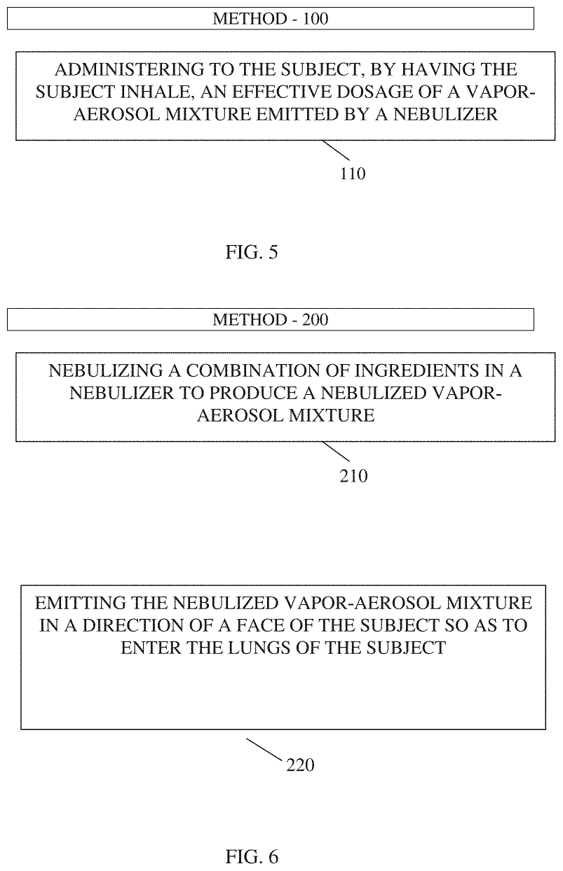

At a molecular level, hydrogenated water moves in a way that generates precession which may be in a clockwise (left to right) direction (i.e. rotation) or may be in a counterclockwise (right to left) direction (i.e. rotation). In certain embodiments, the hydrogenated water nebulized has a counterclockwise spin polarization (also referred to as “right screwed” or “right-handed” or “right to left spin polarization”), which enhances its ability to cause coagulation of the coronavirus spike proteins. In order to obtain right-handed hydrogenated water, the hydrogenated water is first treated (using the same technique used for making the right-handed ethyl alcohol by rotating a magnet near it in a counterclockwise direction. For example, one place the magnet to be rotated directly under a container containing the hydrogenated water. In some implementations, niobium constant magnets are used for this purpose and they have a torsion field in a counter-clockwise direction as shown in FIG. 4. In certain non-limiting implementations, one rotates the magnet at 3000 revolutions per minute for a period of about three minutes. The right-handed or right-screwed hydrogenated water increases the solubility of all ingredients in the mixture and makes it much easier for the ingredients to be absorbed into the epithelial layer of the lungs of the patient/subject/user.

In one particular embodiment, for each milliliter of hydrogenated water placed into the nebulizer 20, one adds between about 0.1 milliliters and about 0.2 milliliters (about 2, 3 or 4 drops), or for example in other embodiments between about 0.05 and about 0.3 milliliters (1 to 6 drops), of approximately 70% ethyl alcohol (or between 40% ethyl alcohol and 80% ethyl alcohol) into the nebulizer 20.

As used herein, the phrase “for each milliliter of hydrogenated water” and the phrase “per milliliter of hydrogenated water” is intended as a relationship or proportion and is not to be understood to be a limitation that requires that there actually be a milliliter of the hydrogenated water present. For example, a statement that there is between 0.1 to 0.2 milliliters of ethyl alcohol for each milliliter of hydrogenated water means that if for example there is three-quarters of a milliliter of hydrogenated water (and not a milliliter) then there should be three-quarters of (0.1 to 0.2), or in other words 0.075 to 0.15 milliliters, of ethyl alcohol.

The 40% to 80% ethyl alcohol is typically in a solution of water. One form of ethyl alcohol is known to cause inebriation. Ethyl alcohol, at the molecular level, moves in a way that generates precession. There is a form of ethyl alcohol that has a left-handed or clockwise spin polarization at the molecular level and there is form of ethyl alcohol that has a right-handed or counterclockwise spin polarization at the molecular level. In certain embodiments, left-handed or clockwise ethyl alcohol is used and in certain embodiments right-handed or counterclockwise ethyl alcohol is used herein in the method, apparatus and/or composition of the invention. Right-handed ethyl alcohol is more effective at generating coagulation for neutralizing the virus but on the other hand it requires some time, for example, 20 minutes, to prepare. Right-handed ethyl alcohol (sometimes called “right-screwed ethyl alcohol” or ethyl alcohol having a right-handed spin polarization) can be prepared by rotating a magnet counterclockwise wherein for example the magnet is situated under or directly under a container holding the ethyl alcohol solution.

In one embodiment, right-handed ethyl alcohol (also referred to herein as right screwed ethyl alcohol because the rotation is like that of a screw) is one of the ingredients placed into the nebulizer 20 to be nebulized. This may be prepared as follows. For embodiments that use right-handed ethyl alcohol, magnets, for example niobium constant magnets 70 (FIG. 4) with a torsion field in a counter-clockwise direction as shown in FIG. 4, are used to prepare the right-handed ethyl alcohol. In some implementations, one would set the rotation of the magnet at 3000 rotations (revolutions) per minute and have it rotate for a period of 3 minutes. In FIG. 4, below the magnet is a motor or other device that generates rotational movement of the magnet.

Right-handed (sometimes called right-screwed) ethyl alcohol is expected to kill the coronavirus (i.e. the virus that may subsequently enter the lungs or that may already be present in the lungs) including by contributing to the coagulation of the spike protein. Vapors of the ethyl alcohol are not expected to cause any harm to the user/subject/patient. Furthermore, if the left-handed (left-screwed) spin polarization ethyl alcohol is used, although it causes inebriation at certain dosages, it will not cause inebriation as used herein because a very low dosage is utilized. In some embodiments, only right screw (right handed) ethyl alcohol is used.

In one particular embodiment of a method (or an apparatus), place between 2 to 4 drops of the ethyl alcohol into the nebulizer 20. In some embodiments, it is right-handed ethyl alcohol that is placed. Using counterclockwise (right-screwed) ethyl alcohol increases the solubility of the mixture and increases penetration of the vapor-aerosol mixture into the virus so as to increase the coagulation effect of the vapor-aerosol mixture on the spike proteins.

In certain embodiments, a storage container 30 of helium, such as a mini-balloon 30 as shown in FIG. 1, is configured adjacent the nebulizer 20 such that the helium is in communication with the nebulizer 20 through an inlet of the nebulizer 20. In some embodiments, the mini-balloon 30 is the type of small balloon used to contain carbon dioxide placed in the rear of the nebulizer 20. The helium may be medical grade helium in communication with the nebulizer 20 through an inlet separate from the outlet 22 of the nebulizer 20 for emitting the VAM.

In some embodiments, for example as shown in FIG. 1, the outlet 22 is integrally formed with an upper portion 23 of the apparatus 10. In certain embodiments, the helium will dilute the nebulized vapors 29 of the other ingredients and limit any potential adverse reactions. In certain embodiments, the helium inlet (not visible in FIG. 1) between the helium storage container 30 and the nebulizer 20 is typically open during the time that the patient inhales the vapor-aerosol mixture 29 from the nebulizer 20. The helium inlet is typically positioned above the expected water level in the nebulizer—in some embodiments the helium inlet is near a top portion of the side wall of the nebulizer 20.

In other embodiments, the helium storage container 30 storing the helium is adjacent the nebulizer 20 but not attached to nebulizer 20. In that case, the helium container is configured to emit the helium so that the helium travels in the same direction as the vapor-aerosol mixture 29 (FIG. 1) towards the patient's face (mouth or nose). For example, the outlet of the helium storage container and the outlet of the nebulizer are oriented such that both emit their respective contents (helium and VAM) to the face of the user from substantially the same direction such that the VAM from the nebulizer and the helium mix together.

Although not shown in FIG. 1, in certain embodiments there is a button or other regulator that regulates the quantity of the helium that flows out of the storage container 30 of the helium and either through the inlet to the inside of the nebulizer 20 or is sprayed in the direction that the vapor-aerosol mixture 29 is emitted by nebulizer outlet 22 (FIG. 1). In one implementation, a relatively small quantity of helium, for example one cc per minute is allowed to be transmitted into the nebulizer 20. The apparatus 10, in certain embodiments, provides the ability to regulate (higher or lower) the amount of the flow of helium.

Nebulizer 20 may also hold an additional (third) ingredient. In some embodiments, this additional ingredient is varied. In order to reduce the risk of the coronavirus developing a resistance to one or two ingredients in the total mixture, it is advantageous in certain embodiments to vary the treatment by varying the third ingredient. In some embodiments, this will ensure that the degree of coagulation of the spike protein does not decline as time goes by while the treatment is being administered. In some implementations of this, one of the variations can be the absence of the third ingredient.

Typically, if there is an additional ingredient, such additional ingredient would be placed into the nebulizer 20 after the water and ethyl alcohol are placed, although this is not a limitation. For this possible additional ingredient there are several options, for example in one embodiment there are five options, although this number is not a limitation. In one embodiment, the first option is not to add an additional component. The second option is to add one drop (or between half a drop and 2 drops) of hydrogen peroxide, for example hydrogen peroxide between 20% to 35%, for example hydrogen peroxide 35%. The third option is to add a low dosage, for example about one quarter of the recently FDA-approved dose of hydroxychloroquine or chloroquine (approved for coronavirus). For example, about 50 mg (or an amount between about 50 mg and about 70 mg) of hydroxychloroquine or chloroquine instead of 200 mgs of hydroxychloroquine or chloroquine. Although hydroxychloroquine or chloroquine is potentially dangerous in rare cases for people with certain heart conditions, the FDA and/or U.S. government has approved it for the use of treating COVID-19 due to the belief that its benefits outweigh its risks.

In accordance with a fourth option for the additional (third) ingredient, in certain embodiments one drop (or at most two drops) (0.05 to 0.1 milliliters, or in other embodiments 0.03 to 0.12 milliliters or in other embodiments 0.025 to 0.12 milliliters or in other embodiments 0.025 to 0.15 milliliters) of the following yellow derivative of an acidified sodium chlorite mixture is added to the nebulizer 20 (per milliliter of hydrogenated water): (i) 28% solution of sodium chlorite NaClO₂ plus an equal amount by volume of (ii) 50% citric acid (half citric acid and half water). When the 28% solution of sodium chlorite and the 50% solution of citric acid are mixed for about thirty seconds, the mixture turns yellow and this yellow derivative is ready for addition (for example in the form of drops) into the nebulizer 20 at a very low dosage. It is noted that NaClO2 has been approved by FDA for certain uses including washing food. Furthermore, extremely low dosages of NaClO2 are being used in the prevention and/or treatment herein. Furthermore, the mixture of sodium chlorite and citric acid is mixed externally to the nebulizer 20. Any dangerous chlorine dioxide that could result from the mixture evaporates since chlorine dioxide exists as a gas above 11 degrees Centigrade. The remaining liquid is what is used for the additional ingredient to be placed in the nebulizer 20 in the fourth option. The benefits of using the yellow derivative of the acidified sodium chlorite mixture in very low dosages in vaporized form may outweigh the risks under certain circumstances such as a pandemic when used carefully. However, in the event the inclusion of this ingredient is not approved in a particular jurisdiction (such as the U.S.) it would not be used.

The term “per milliliter of hydrogenated water” in this case means that if for example about three-quarters of a milliliter of hydrogen water were used, then about three quarters of the 0.05 to 0.1 milliliters, or in other embodiments of the 0.03 to 0.12 milliliters of the sodium chlorite mixture is used. This term “per milliliter of hydrogenated water” does not imply a requirement that there actually be a milliliter of hydrogenated water placed into the nebulizer 20.

In certain embodiments, use of more than a milliliter of hydrogenated water will not result in a proportionately greater dosage of sodium chlorite but rather the proposed range of for 0.03 to 0.12 milliliters may be viewed as an upper limit.

In accordance with a fifth option, other ingredients are added such as antibiotics (in one non-limiting example Azithromycin) are added to the nebulizer 20 to be nebulized in an amount of about 50 mgs (which is about one tenth the regular recommended dosage, for example between 30 and 150 mgs. In one version of the fifth option, essential oils are added.

As shown in FIG. 1, in certain embodiments the nebulizer apparatus 10 includes a storage container 30 of helium, such as a mini-balloon 30, in communication with the nebulizer 20 such as through an inlet. This allows the inside of the nebulizer 20 to be in communication with medical helium for example in gas form. In some embodiments, one can take an existing nebulizer 20 and making an aperture in nebulizer 20 for an inlet to a helium storage container 30 such as a mini-balloon 30 so that the inside of the nebulizer 20 is in communication with helium gas (like the helium used in balloons). The helium is useful so that when the user inhales (by breathing) the vapor-aerosol mixture 29 (VAM) emitted by nebulizer 20, the helium causes the partial pressure of the nebulized mixture 29 to increase, which in turn generates increased penetration of the ingredients in the mixture 29 into the patient's alveoli. The pressure of the air (together with the vapor-aerosol mixture) going into lungs is higher due to the pressure of the helium and the helium thereby draws with it into the cell the vapors of the hydrogenated water and ethyl alcohol and that of the sodium chlorite mixture (if present) or other ingredient. The helium is inert and does not otherwise react with the other ingredients of the vapor-aerosol mixture 29.

In use, the user adds all of the ingredients to the nebulizer 20 and turns on the nebulizer 20, which typically operates using ultrasound technology. In general, all of the ingredients (hydrogenated water, ethyl alcohol plus any third ingredient) are nebulized together. Accordingly, the user/patient/subject inhales the nebulized vapor-aerosol mixture 29 as a unit in certain embodiments.

As shown in FIG. 1, nebulizer 20 includes piezoelectric crystals 25 that are used to generate suitable frequencies (2.5 Mhz-35 Mhz), for example suitable ultrasound frequencies, to produce suitable sizes of aerosol micro-particles. In case of an additional ingredient being antibiotics (or sodium chlorite or hydroxychloroquine or chloroquine or hydrogen peroxide), for example Azithromycin antibiotics, using a nebulizer that has piezoelectric crystals having a high frequency within the range of 2.5 MHz to 35 MHz so as to produce aerosol micro-particles of from 0.25 microns to 5 microns allows the antibiotics (or other ingredient) to more easily penetrate the cells of the subject.

In use, a vapor-aerosol mixture 29 (“VAM”) is generated by the nebulizer 20. As shown in FIG. 1, the patient or subject inhales the nebulized vapor-aerosol mixture 29 from the nebulizer 20 for about 5-15 minutes, in one non-limiting example about ten minutes, in another non-limiting embodiment about 15 minutes or 10-20 minutes or 12-18 minutes or 8-12 minutes or ten minutes. In some embodiments this is repeated three times a day, for example in the morning, the afternoon and the evening.

In certain embodiments, the nature of the ingredients used in the prevention and/or treatment is varied in order to increase the influence of the vapor-aerosol mixture 29 on the spike protein and to prevent resistance to one or two ingredients from developing. In one particular non-limiting example, the first day, option 1 (no additional ingredient) is used, the second day option 2 (hydrogen peroxide) is used as the additional (third) ingredient along with the hydrogenated water and ethyl alcohol, the third day option 3 (hydroxychloroquine or chloroquine) is used as the additional ingredient and the fourth day option 4 (sodium chlorite) is used as the additional ingredient. Then the fifth day option 5 (antibiotics) is used as the additional ingredient. The on the sixth day option 1 (no additional ingredient) is used and on the seventh day option 2 (hydrogen peroxide) is used as the additional ingredient and so on and so on. In an alternative implementation of the varying of the additional ingredient, the changes occur are after each treatment as opposed to after each day (the term “treatment” used broadly to encompass prevention). In that implementation, option 1 is used in the morning, option 2 in the afternoon treatment and option 3 in the evening treatment and option 4 is used for the next morning's treatment and option 5 is used in the afternoon and so on. Other variations are possible.

In certain embodiments, this treatment is given to a healthy person for 10 days or 2 weeks or 3 weeks or for as long as the person is in danger of being exposed to others who test positive for coronavirus (such as COVID-19).

In certain embodiments, this treatment is given to a healthy person for 3-5 days (or in other embodiments 3-7 days 3 days or 4-6 days) after that person has come into contact with another person who has tested positive for coronavirus (such as COVID-19).

In certain embodiments, this treatment is applied for 2 weeks or 3 weeks or until a negative coronavirus (such as COVID-19) test result is reached, or for example until cessation of symptoms. In some embodiments, treatment is applied for as long as a pandemic or epidemic is present in the environment of the individual subject. In certain embodiments the treatment is applied for however long the subject is expected to be at risk of exposure to the coronavirus (such as COVID-19).

Use of the treatment will cause the virus to die because the vapors of the ethyl alcohol (especially right screwed or right handed ethyl alcohol) in combination with that of the hydrogenated water (especially right screwed or right handed hydrogenated water) will cause coagulation of the spike proteins, and this effect is enhanced by delivery aided by helium (in certain embodiments) and is enhanced by the additional ingredient, for example sodium chlorite.

In addition, in certain embodiments, the use of the treatment will also cause coagulation of the ACE2 protein. In that case, the concentration of ACE2 will be very low, at least temporarily.

If the person who takes the treatment is later exposed to the coronavirus, the spike protein of the virus that he or she inhales will not be able to penetrate the cells of the lungs of the person because the spike protein will be coagulated and neutralized such that the virus is effectively dead. When the spike protein is coagulated, it will be unable to bind with the ACE2 of the cell membrane of the person/subject/patient. In some cases, the ACE2 of the person's epithelial lung cells will also coagulate and further inhibit any potential binding between the spike protein and the lung cells. The virus will not penetrate the lungs and will be rendered harmless even after the person inhales air containing the virus.

In certain embodiments, use of the treatment results in coagulation of the envelope of the coronavirus (for example COVID-19), i.e. coagulation of the spike proteins of the coronavirus which results in the virus' loss of ability to attach to the normal epithelial cells of lungs alveolus.

In some embodiments, the treatment is administered to a mammalian subject, such as a human individual, at a time when the individual is already infected with coronavirus, for example COVID-19. In that case, the treatment is initiated at a time when the virus has already penetrated cells, for example epithelial lung cells. Binding of the spike protein to the ACE2 has already occurred. In that case, certain embodiments of the method and/or apparatus herein is still helpful because the VAM from the nebulizer apparatus 10 enters the subject's lung cells and does not allow the RNA of virus to replicate further because the VAM coagulates the RNA. Therefore, the presence of the virus in patients who are already infected will be reduced as a result of the treatment. As a result, it is expected that such infected patients will test negative for coronavirus, for example COVID-19, much faster.

As a result of the death of the virus (when the spike protein is coagulated) the lungs will be cleaned out (whether the subject is healthy or infected) The alveolar epithelial cells of the lungs become clear. Superficial mucous of the alveolar epithelial cells is created and this drains out normally.

After use of the treatment in certain embodiments, the coronavirus, for example COVID-19, has now become harmless because of the spike protein coagulation and/or coagulation of the ACE2 protein and the spike protein cannot bind to the ACE2 protein. In some case, also because of the coagulation of the ACE2 enzyme the binding is further inhibited. Applicant has used the treatment for over two weeks consecutively and has suffered no side effects.

In another embodiment, a population of individuals are given the treatment to prevent the spread of the coronavirus pandemic amongst that population.

One embodiment, a method 100 of preventing infection of a mammalian subject by a coronavirus, comprises a step 110 of administering to the subject, by having the subject inhale, an effective dosage of a vapor-aerosol mixture emitted by a nebulizer. The effective dosage in some embodiments is derived from the following quantities placed into the nebulizer: (wherein in some embodiments there is at least half a milliliter of hydrogenated water), for each milliliter of hydrogenated water, ethyl alcohol between 40% and 80% comprising ethyl alcohol, wherein at least one of the ethyl alcohol and hydrogenated water has a right-handed spin polarization. The ethyl alcohol or hydrogenated water may have a right-handed or right-screwed or counterclockwise spin polarization, for example as a result of being treated with a counterclockwise rotating magnet. The hydrogenated water may be right handed or right screwed or counterclockwise hydrogenated water and may comprise 1 milliliter (plus or minus 20%). The absolute amount of hydrogenated water is not essential as long as the proportion between the hydrogenated water, ethyl alcohol and additional ingredient (sodium chlorite or hydroxychloroquine or chloroquine or hydrogen peroxide or antibiotics or essential oil) is maintained (subject to not exceeding the maximum allowable amount of sodium chlorite).

In one implementation of the method, the mixture also contains one of the following (a) hydrogen peroxide, (b) hydroxychloroquine or chloroquine, (c) mixture of sodium chlorite with food-grade citric acid solution, (d) antibiotics. In one implementation, the mixture may also contain essential oils or other suitable additional ingredients. The hydroxychloroquine or chloroquine is between 25% and 35% of a governmentally approved dosage of the hydroxychloroquine or chloroquine for each milliliter of hydrogenated water in the nebulizer 20. For example, the hydroxychloroquine or chloroquine is 50 mgs (the recommended dosage being 200 mgs) or from 50 mgs to 70 mgs) for a nebulizer 20 in which about one milliliter of hydrogenated water is used. The mixture of sodium chlorite with food-grade citric acid solution is between about 0.05 milliliters and about at most 0.1 milliliters (for example 1 small drop or at most two small drops), or in some other embodiments between 0.03 and 0.1 milliliters, for nebulizer 20, in a case in which about one milliliter of hydrogenated water is placed in the nebulizer 20.

In one implementation of the method, a flow of helium is provided during the inhalation of the VAM emitted by the nebulizer either as a result of the inside of the nebulizer 20 being in communication, such as through an inlet in the nebulizer 20, with a storage container 30 of helium such as a mini-balloon 30 (FIG. 1), or as a result of the storage container 30 of helium being positioned, for example behind the nebulizer 20 and not integrally attached to the nebulizer 20, so as to direct pressurized helium (for example having a pressure of about a quarter to a half of an atmosphere) in the same direction as the flow of vapor-aerosol mixture emitted by the nebulizer 20 such that the two flows merge and the helium delivers the VAM into the lungs more forcefully than without the helium.

In some embodiments, the administering is performed such that the mixture to be nebulized also contains one of (a) hydrogen peroxide, (b) hydroxychloroquine or chloroquine, (c) mixture of sodium chlorite with food-grade citric acid solution, (d) antibiotics, wherein the regimen varies daily or varies with each treatment with respect to a) hydrogen peroxide, (b) hydroxychloroquine or chloroquine, (c) mixture of sodium chlorite with food-grade citric acid solution, (d) antibiotics (e) another ingredient such as essential oils.

In the administering, the mixture held by the nebulizer to be nebulized also may vary the third ingredient, i.e. the one of the following: (a) hydrogen peroxide, (b) hydroxychloroquine or chloroquine, (c) mixture of sodium chlorite with food-grade citric acid solution, (d) antibiotics, daily or with each treatment until each of the four options has been selected. In order to reduce the risk of the coronavirus developing a resistance to one or two ingredients in the mixture 29, it is advantageous in certain embodiments to vary the treatment by varying the third ingredient (either daily or with each treatment or in another interval).

Another embodiment is an apparatus 10 configured for preventing infection of a mammalian subject by coronavirus by administering a VAM dosage to the subject. The apparatus 10 comprises a nebulizer 20 holding ethyl alcohol and hydrogenated water, the nebulizer 20 configured to nebulize into a vapor-aerosol mixture 29 the ethyl alcohol and the hydrogenated water. In one embodiment, one or both of the ethyl alcohol and hydrogenated water has a counterclockwise spin polarization. The nebulizer 20 may also receive and contain, and is configured to nebulize into the vapor-aerosol mixture 29, one of the following (a) hydrogen peroxide, (b) hydroxychloroquine or chloroquine (c) mixture of sodium chlorite with food-grade citric acid solution, (d) antibiotics. The nebulizer apparatus 10 may also have a storage container 30 of helium (which may be a balloon or mini-balloon) such that the helium is in communication with an inside of the nebulizer 20 for example through an inlet of the nebulizer 20, as shown in FIG. 1. The storage container 30 may instead be a balloon or mini-balloon adjacent to but not integrally attached to the nebulizer 20 provided the direction of the helium is configured such that the helium emitted mixes with, and helps to thrust, the vapor-aerosol mixture 29 (emitted from nebulizer 20) into the user's subject's lungs. Any hydroxychloroquine or chloroquine present in the nebulizer 20 is in some versions in an amount of 50-70 mgs, or for example between 25% and 35% of a governmentally approved dosage of the hydroxychloroquine or chloroquine (which is about 50 mgs) for a nebulizer in which the hydrogenated water is about 1 milliliter (0.8 to 1.2 milliliter). In other versions, any hydroxychloroquine or chloroquine present in the nebulizer 20 is in an amount of 45-75 mgs or 40-80 mgs. Any mixture of sodium chlorite with food-grade citric acid solution that is present in the nebulizer may be between 0.05 and 0.1 milliliters, which may be implemented through 1 or 2 small drops, or between 0.03 and 0.1 milliliters.

There may be between 0.1 and 0.2 milliliters (or in other embodiments between 0.05 and 0.3 milliliters) of the ethyl alcohol (for example implemented through 2-4 drops or 1-6 drops, each of the drops being about one-twentieth of a milliliter) in the nebulizer 20 for each approximately 1 milliliters of hydrogenated water.

A further embodiment of the apparatus 10 configured for treating a mammalian subject to prevent infection by coronavirus, the apparatus 10 (FIG. 1) comprising a nebulizer 20 holding ethyl alcohol and hydrogenated water and configured to nebulize into a vapor-aerosol mixture 29 the ethyl alcohol and the hydrogenated water, wherein the nebulizer 20 has piezoelectric crystals 25 configured to generate a frequency ranging from a low frequency of below 7 MHz (for example 2.5 MHz) to a high frequency above 30 MHz (for example 35 MHz) so as to produce sizes of aerosol micro-particles of between 0.25 microns and 5 microns in the vapor-aerosol mixture 29.

A further embodiment is a composition comprising a nebulized vapor-aerosol mixture produced by nebulizing a combination of ingredients, the mixture under pressure to move through the air, the combination comprising hydrogenated water and ethyl alcohol and having a proportion such that for each milliliter of hydrogenated water in the combination, there is between 0.05 and 0.3 milliliters of ethyl alcohol of a solution between 40% and 80% ethyl alcohol, wherein at least one of the hydrogenated water and the ethyl alcohol has a counterclockwise spin polarization. There is no requirement that there actually be a milliliter of hydrogenated water so that if there happens to be half a milliliter then there is half of somewhere between 0.05 and 0.3 milliliters of ethyl alcohol.

In some implementations of this embodiment, the hydrogenated water has a counterclockwise spin polarization. The combination may further include, per milliliter of hydrogenated water (without increasing the amount of sodium chlorite), one of the following: (i) between 0.03 and 0.1 milliliters of a mixture of sodium chlorite with citric acid solution (not to exceed this amount even if the amount of hydrogenated water exceeds a milliliter); (ii) 50-70 mgs of hydroxychloroquine or chloroquine; (iii) 0.025 to 0.1 milliliters of hydrogen peroxide that is between 20% and 35% hydrogen peroxide; (iv) between 30 mg and 150 mg of an antibiotic. The nebulized vapor-aerosol mixture may also be under pressure to move from helium under pressure adjacent the mixture, wherein the helium flows into the inside of the nebulizer through an inlet or else flows in the same direction toward the user/patient/subject as the direction of the vapor-aerosol mixture emitted by the nebulizer.

Another embodiment is a method 200 of delivering a combination of ingredients directly to the lungs of a live mammalian subject, comprising a step 210 of nebulizing the combination of ingredients in a nebulizer to produce a nebulized vapor-aerosol mixture, the combination having a proportion such that for each milliliter of hydrogenated water in the combination, there is between 0.05 and 0.3 milliliters of ethyl alcohol of a solution between 40% and 80% ethyl alcohol, wherein at least one of the hydrogenated water and the ethyl alcohol has a counterclockwise spin polarization. Another step 220 of the method 200 is emitting the nebulized vapor-aerosol mixture in a direction of a face of the subject so as to enter the lungs of the subject.

As a result of the method 200 of direct delivery, the dosages of the ingredients are low dosages since they go directly to the lungs. In some embodiments, the delivered combination of ingredients coagulates and thereby neutralizes the spike proteins of a coronavirus that later enters the alveoli of the lungs (or already entered) and in some embodiments coagulates the ACE2 protein of the lung cells.

In some implementations of the method 200, the method 200 further comprises providing a thrust of helium, the helium under pressure in a storage container 30, in a particular direction so as to mix with the vapor-aerosol mixture and drive the vapor-aerosol mixture in the direction of the face of the user. In some cases, the storage container is in communication with an inside of the nebulizer. In some versions, the combination of ingredients is any of the combination of ingredients described herein for any method, apparatus or combination described herein. For example, the combination may further include for each milliliter of hydrogenated water in the nebulizer (this is a proportion and does not require the actual presence of the whole milliliter of hydrogenated water necessarily), one of the following: (i) between 0.025 and 0.15 milliliters of a mixture of sodium chlorite with citric acid solution; (ii) 40-80 mgs of hydroxychloroquine or chloroquine; (iii) 0.025 to 0.1 milliliters of hydrogen peroxide that is between 20% and 35% hydrogen peroxide; (iv) between 30 mg and 150 mg of an antibiotic, or any other combination of ingredients described herein for any method, apparatus or combination described herein.

While the invention has been described with respect to a limited number of embodiments, it will be appreciated that the above descriptions are intended only to serve as examples, and that many other embodiments are possible within the scope of the present invention as defined in the appended claims and that many variations, modifications and other applications of the invention may be made. Therefore, the claimed invention as recited in the claims that follow is not limited to the embodiments described herein.

1. A method of preventing infection of a mammalian subject by a coronavirus, comprising:
administering to the subject, by having the subject inhale, an effective dosage of a vapor-aerosol mixture emitted by a nebulizer derived from the following quantities placed into the nebulizer:
hydrogenated water, and
ethyl alcohol between 40% and 80%, wherein at least one of the ethyl alcohol and hydrogenated water has a counterclockwise spin polarization.

2.-10. (canceled)

11. An apparatus configured for use in prevention and/or treatment of an infection of a human subject by a coronavirus, the apparatus comprising:
a nebulizer holding a mixture of
(a) ethyl alcohol, the ethyl alcohol being a solution of between 40% and 80% ethyl alcohol; and
(b) hydrogenated water,
wherein the mixture contains 0.05 milliliters to 0.3 milliliters of the ethyl alcohol for each milliliter of the hydrogenated water,
the nebulizer configured to nebulize the mixture into a vapor-aerosol mixture of the ethyl alcohol and the hydrogenated water,
wherein (ii) at least one of the ethyl alcohol and the water has a counterclockwise spin polarization.

12. The apparatus of claim 11, wherein the mixture also includes one of the following (a) hydrogen peroxide, (b) hydroxychloroquine or chloroquine (c) a yellow derivative of a mixture of sodium chlorite with citric acid solution, (d) antibiotics.

13. The apparatus of claim 11, further comprising a storage container of helium such that the helium is configured to be emitted from the storage container in a direction so as to mix with the vapor-aerosol mixture emitted from the nebulizer and jointly enter a lungs of a user.

14. The apparatus of claim 11, further comprising a storage container of helium such that the helium is in communication with the nebulizer through an inlet of the nebulizer.

15. The apparatus of claim 14, wherein the storage container is a balloon adjacent the nebulizer.

16. The apparatus of claim 14, wherein the mixture also includes one of the following (a) hydrogen peroxide, (b) hydroxychloroquine or chloroquine (c) a yellow derivative of a mixture of sodium chlorite with citric acid solution, (d) antibiotics.

17. The apparatus of claim 16, wherein the hydroxychloroquine or chloroquine is between 50 and 70 mgs for each milliliter of hydrogenated water in the nebulizer.

18. The apparatus of claim 16, wherein a quantity of the yellow derivative of the mixture of sodium chlorite with citric acid solution is between 0.03 and 0.1 milliliters.

19.-20. (canceled)

21. The apparatus of claim 11, wherein the mixture includes 50-70 mgs of hydroxychloroquine or chloroquine.

22. The apparatus of claim 11, wherein the mixture includes between 0.03 and 0.1 milliliters of a yellow derivative of a mixture of sodium chlorite with citric acid solution in the nebulizer.

23. A nebulized vapor-aerosol mixture produced by nebulizing a combination of ingredients, the mixture under pressure to move through the air, the combination comprising hydrogenated water and ethyl alcohol and having a proportion such that for each milliliter of hydrogenated water in the combination, there is between 0.05 and 0.3 milliliters of ethyl alcohol of a solution of between 40% and 80% by volume ethyl alcohol, wherein at least one of the hydrogenated water and the solution of ethyl alcohol has a counterclockwise spin polarization.

24. The nebulized vapor-aerosol mixture of claim 23, wherein the hydrogenated water has a counterclockwise spin polarization.

25. The nebulized vapor-aerosol mixture of claim 23, wherein the combination further includes for each milliliter of hydrogenated water, one of the following:
(i) between 0.03 and 0.12 milliliters of a derivative of a mixture of sodium chlorite with citric acid solution;
(ii) 40-80 mgs of hydroxychloroquine or chloroquine;
(iii) 0.025 to 0.1 milliliters of hydrogen peroxide that is between 20% and 35% hydrogen peroxide;
(iv) between 30 mg and 150 mg of an antibiotic.

26. The nebulized vapor-aerosol mixture of claim 23, wherein the mixture is also under pressure to move from helium under pressure adjacent the mixture.

27. A method of delivering a combination of ingredients directly to the lungs of a live mammalian subject, comprising:
nebulizing the combination of ingredients in a nebulizer to produce a nebulized vapor-aerosol mixture, the combination having a proportion such that for each milliliter of hydrogenated water in the combination, there is between 0.05 and 0.3 milliliters of ethyl alcohol of a solution between 40% and 80% ethyl alcohol, wherein at least one of the hydrogenated water and the ethyl alcohol has a counterclockwise spin polarization; and
emitting the nebulized vapor-aerosol mixture in a direction of a face of the subject so as to enter the lungs of the subject.

28. The method of claim 27, further comprising providing a thrust of helium, the helium under pressure in a storage container, in a particular direction so as to mix with the vapor-aerosol mixture and drive the vapor-aerosol mixture in the direction of the face of the user.

29. (canceled)

30. The method of claim 27, wherein the combination further includes for each milliliter of hydrogenated water in the nebulizer, one of the following:
(i) between 0.025 and 0.15 milliliters of a derivative of a mixture of sodium chlorite with citric acid solution;
(ii) 40-80 mgs of hydroxychloroquine or chloroquine;
(iii) 0.025 to 0.1 milliliters of hydrogen peroxide that is between 20% and 35% hydrogen peroxide;
(iv) between 30 mg and 150 mg of an antibiotic.

31. The apparatus of claim 11, further comprising a vapor-aerosol mixture of the ethyl alcohol and the hydrogenated water.

32. The apparatus of claim 31, wherein the vapor-aerosol mixture is situated (i) inside the nebulizer, wherein inside the nebulizer includes space defined by an outlet of the nebulizer, (ii) outside the nebulizer or (iii) partly inside and partly outside, the nebulizer.

33. The apparatus of claim 31, wherein the vapor-aerosol mixture is situated outside the nebulizer.

34. The apparatus of claim 31, wherein the vapor-aerosol mixture is under pressure to move through air.

35. The apparatus of claim 31, wherein an amount of the water exceeds an amount of the ethyl alcohol.

36. The apparatus of claim 31, wherein the ethyl alcohol is in a solution of between 40% and 80% ethyl alcohol and an amount of the hydrogenated water exceeds an amount of the solution of ethyl alcohol.

37. A kit configured for use in prevention and/or treatment of an infection of a mammalian subject by a coronavirus, the kit comprising:
(a) a nebulizer configured to nebulize contents of the nebulizer into a vapor-aerosol mixture;
(b) ingredients configured to be placed into the nebulizer, wherein the ingredients comprise water and ethyl alcohol, wherein at least one of the water and ethyl alcohol has a counterclockwise spin polarization.

38. The kit of claim 37, wherein the water is hydrogenated water.

39. The kit of claim 37, wherein each of the water and ethyl alcohol has a counterclockwise spin polarization.

40. The kit of claim 37, wherein the ethyl alcohol is in a solution of between 40% and 80% ethyl alcohol and an amount of the water exceeds an amount of the solution of ethyl alcohol.
